(19) United States
(12) Patent Application Publication
(10) Pub. No.: US 2005/0010183 A1
(43) Pub. Date: Jan. 13, 2005

(54) Absorbent structure for absorbing blood

(75) Inventors: Charles E. Miller, Weatogue, CT (US); Michael R. Hansen, Lynnwood, WA (US)

Correspondence Address: WEYERHAEUSER COMPANY;INTELLECTUAL PROPERTY DEPT., CH 1J27, P.O. BOX 9777, FEDERAL WAY, WA, 98063, US

(73) Assignee: Weyerhaeuser Company

(21) Appl. No.: 10/603,981

(22) Filed: Jun. 24, 2003

(51) Int. Cl. B32B 27/04; D04H 3/00; B32B 27/12; D04H 1/00; B32B 5/02; A61F 13/15; A61F 13/20

(52) U.S. Cl. 604/367; 442/123; 442/121; 442/414; 442/415; 442/381; 442/392; 442/389

(57) ABSTRACT

Absorbent structures for use in articles for absorbing blood include a blood absorbent enhancing agent that is present in a non-uniform distribution throughout the absorbent structure.

### FIELD OF THE INVENTION

The present invention relates to structures for absorbing blood such as are included in feminine hygiene products, e.g., sanitary napkins.

### BACKGROUND OF THE INVENTION

In the past, attempts have been made to design feminine hygiene products having improved absorbent utilization and controlled staining and flow characteristics. Feminine hygiene products are traditionally made from cellulosic material. While these materials provide absorbency when used without modification, they suffer from certain disadvantages. For example, menstrual fluid is not immobilized in wood pulp fluff or other conventional cellulosic absorbent materials. Accordingly, when pressure is exerted which compresses these absorbents, the fluid is liberated and can either be pushed upward through a fluid permeable covering to contact the wearer or can run outward along the top surface of the absorbent and may promote side staining.

Attempts have been made to at least partially immobilize menstrual fluid and increase the absorptive capacity of feminine hygiene products by the inclusion of superabsorbent materials. The superabsorbent material is generally in the form of a crosslinked three-dimensional structure, which is designed to allow for the penetration of an aqueous fluid. However, the suspended plasma proteins and blood cells in menstrual fluid are too large to penetrate into the superabsorbent material and tend to surround the superabsorbent material, thereby adversely affecting its ability to absorb the liquid fraction of the menstrual fluid.

The interaction of blood with superabsorbent material is further complicated in that the blood cells and plasma proteins present in menstrual fluid have positively charged surfaces, while the superabsorbent material has a negatively charged surface. Thus, by virtue of electrostatic attractive forces, the superabsorbent material tends to bind a small portion of the solids fraction of the menstrual fluid and become impervious to liquid penetration. Thus, a balancing equilibrium is determined in any particular feminine hygiene product by the relative magnitude of the affinities of the superabsorbent materials and the blood solids for the aqueous portion of the blood as well as the relative absolute amounts of each component.

While superabsorbent material initially absorbs the aqueous portion of blood containing fluids with extreme rapidity, when the protein fraction of the blood surrounds the individual superabsorbent particles as described above, the absorption of the fluid as a whole is stopped before any significant amount of the fluid is contained within the superabsorbent particles.

Performance characteristics of feminine hygiene products, which ultimately affect user acceptance, include the rate at which the absorbent structure absorbs the menstrual fluid. Absorbent structures that absorb the menstrual fluid more quickly are generally considered more desirable than those that absorb the menstrual fluid at a slower rate. Another factor considered in the performance of a feminine hygiene product is its capacity to absorb menstrual fluid. Generally, larger capacities are more desirable because they translate into longer wearing times or a diminished probability of leakage. Absorbency under load (AUL) is another important performance criteria. Absorbency under load is an indicator of the ability of a feminine hygiene product to retain absorbed menstrual fluid when pressure is applied to the product. Feminine hygiene products, which have a higher absorbency under load, are less likely to leak when compressed as compared to feminine hygiene products that have lower absorbency under load characteristics.

### SUMMARY OF THE INVENTION

The present invention provides absorbent structures capable of absorbing blood rapidly and retaining it once absorbed. The structures are useful in feminine hygiene products such as sanitary napkins and other structures that absorb blood, such as bandages and surgical towels.

In one embodiment of the present invention, an absorbent structure is provided that includes a first web of fibers having a first surface and a second surface that is spaced from the first surface. The web of fibers includes a blood absorbent enhancing agent that promotes coagulation of the dissolved and suspended solids in the blood. The blood absorbent enhancing agent is present in the web in a first amount adjacent the first surface and a second amount adjacent the second surface, wherein the first amount is unequal to the second amount. Accordingly, absorbent structures formed in accordance with this embodiment of the present invention include a non-uniform distribution of the blood absorbent enhancing agent throughout the web. These webs can be combined with other webs of fibers that serve functions such as an acquisition layer or layers for retaining the absorbed fluids.

In another embodiment, the present invention relates to an absorbent structure that includes a first web that includes fibers that are bonded together. The first web has a first density. The absorbent structure further includes a second web comprising fibers and a blood absorbent enhancing agent. In this embodiment, the amount of the blood absorbent enhancing agent in the second web may be non-uniform as described above.

The absorbent structures of the present invention exhibit acquisition, retention and capacity characteristics that make them desirable alternatives to existing materials used in commercially available blood absorbent products such as sanitary napkins.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic illustration of a cross-section through a first aspect of a first embodiment of the present invention;

FIG. 2 is a schematic illustration of a cross-section through a second aspect of the first embodiment of the present invention;

FIG. 3 is a schematic view of a cross-section through the first embodiment of the present invention with an additional superabsorbent material containing layer illustrated;

FIG. 4 is a schematic illustration of a cross-section through an absorbent structure formed in accordance with a second embodiment of the present invention;

FIG. 5 is a schematic illustration of a test apparatus for characterizing the absorbency under load and capacity characteristics of an absorbent structure; and

FIG. 6 is a graph illustrating the absorbency under load and retention characteristics of the samples evaluated in Example 1.

### DETAILED DESCRIPTION OF THE PREFERRED EMBODIMENTS

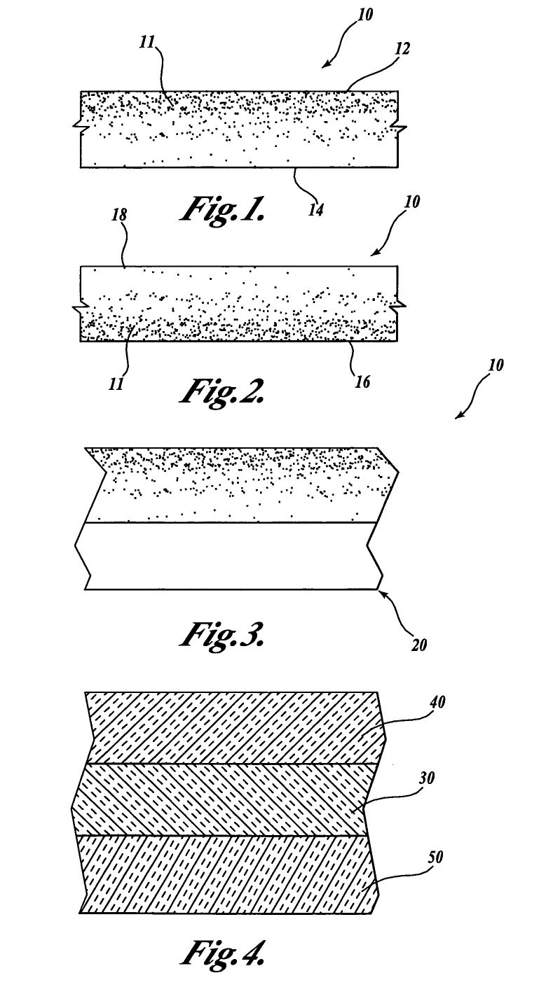

Referring to FIGS. 1 and 2, in a first aspect of the present invention, a web 10 of fibers includes a non-uniform distribution of a blood absorbance enhancing agent, schematically illustrated by the shading 11. In the structure illustrated in FIG. 1, the amount of the blood absorbent enhancing agent is greater near a top surface 12 of web 10 as compared to the amount of blood absorbent enhancing agent adjacent bottom surface 14. In an alternative embodiment illustrated in FIG. 2, the amount of blood absorbent enhancing agent is greater near bottom surface 16 as compared to the amount of blood absorbent enhancing agent adjacent top surface 18.

Web 10 is a mass of fibers that can be formed by conventional techniques such as airlaying. When the fibers are cellulose fibers, an airlaid web can be made by individualizing a cellulose pulp sheet in a hammer mill. The individualized cellulose fibers are then entrained in an air stream. The entrained fibers are then delivered to a porous support where they are deposited. Airlaying of cellulose fibers and other fibers is well known to those skilled in the art, and accordingly, further description of such process is not necessary herein.

The density of the web 10 can vary depending upon the ultimate end use. For applications in feminine hygiene products such as sanitary napkins, densities can range from about 0.03 to about 0.6 g/cm³.

Web 10 can be formed from many different types of fibers that are known to be useful in absorbent structures. Such fibers include synthetic fibers such as acrylic, polyester, carboxylated polyolefins, rayon, polyethylene, polypropylene, multicomponent fibers, and nylon. These fibers may be thermobondable (fibers that bind to each other upon heating) or they may be treated with bonding agents capable of binding the fibers to each other. Suitable examples of fibers that have been treated with bonding agents to bind them together are described in U.S. Pat. Nos. 5,057,166 and 5,837,627, the teachings of which regarding thermobondable fibers and fibers treated with bonding agents are incorporated expressly herein.

The fibers of web 10 can also be cellulose fibers such as wood pulp fibers. The wood pulp fibers may be chemical, mechanical, chemimechanical, thermomechanical or chemithermomechanical, or combinations thereof. Suitable wood pulp fibers include those obtained from well known chemical processes such as the kraft and sulfite processes. Ground wood fibers, recycled, or secondary wood pulp fibers, and bleached and unbleached wood pulp fibers can be used. Details of the production of wood pulp fibers are well known to those skilled in the art. These fibers are commercially available from a number of companies, including Weyerhaeuser Company.

Other examples of useful fibers include chopped silk fibers, bagasse, hemp, jute, rice, wheat, bamboo, corn, sisal, cotton, flax, kenaf, peat moss, and mixtures thereof. In addition, blends of synthetic and cellulose-type fibers can be used.

The cellulose fibers may be pretreated prior to combination with the blood absorbent enhancing agents in accordance with the present invention. Such pretreatment may include physical treatment, such as subjecting the fibers to steam, or chemical treatment, such as crosslinking the fibers. Although not to be construed as a limitation, examples of pretreating fibers also include the application of chelaters, deodorants, and antimicrobials, such as by spraying the fibers with such materials. In addition, the fibers may be pretreated with surfactants or other liquids, such as water or solvents, which may modify the surface of the fibers. Other pretreatments include exposure to fragrances or pigments. The fibers also may be pretreated in a way that increases their wettability or they may be pretreated with chemicals that result in lignin or cellulose-rich fiber surfaces.

Bleaching processes, such as chlorine or ozone/oxygen bleaching may also be used in pretreating the fibers. The cellulose fibers may also be pretreated with thermoplastic and thermoset resins.

The blood absorbent enhancing agent that is applied to the fibers in accordance with the present invention include those types of blood absorbent enhancing agents described in U.S. Pat. No. 5,998,032, the subject matter of which is expressly incorporated herein by reference. The blood absorbent enhancing agents are capable of promoting the coagulation of the dissolved and suspended solids in the blood. In this manner, the dissolved and suspended solids are to some extent separated from the aqueous components of the blood. This coagulation tends to immobilize the solid fraction of the blood and facilitate the wicking distribution of the remaining liquid fraction. Also, by separating portions of the dissolved solids from the aqueous components, the adverse effect the dissolved solids components have on the ability of superabsorbent materials to absorb the aqueous portions is reduced. In addition, the mobility of the dissolved and suspended solids after being separated from the aqueous components is reduced, making them more susceptible to retention within the webs of the present invention. The enhancing agents are generally organic and have at least one hydrogen bonding functionality. Generally, such blood absorbent enhancing agents can be broken down into polymeric and non-polymeric agents. The polymeric enhancing agents generally include molecules that have at least one hydrogen bonding functionality or coordinate covalent bond forming functionality. Examples of suitable polymeric blood absorbent enhancing agents include repeating units, wherein the repeating units have functionality on each repeating unit of the polymer, although this is not necessary for adequate blood absorbent enhancing agent properties. Exemplary polymeric blood absorbent enhancing agents include polyglycols, especially polypropylene glycol; polycarboxylic acids; polycarboxylates; poly(lactone)polyols, such as diols; polyamides; polyamines; polysulfonic acids; polysulfonates; and combinations thereof. Some specific examples of these types of polymeric agents include the polyglycols, polypropylene glycol, and polyethylene glycol; the poly(lactone)polyols, such as poly(caprolactone)diol and poly(caprolactone)triol; polycarboxylic acids, such as polyacrylic acid and polymaleic acid; polyamides such polyacrylamide or polypeptides; polyamines such as polyethylenimine and polyvinylpyridine; polysulfonic acids or polysulfonates such as poly(sodium-4-styrenesulfonate) or poly(2-acrylamido-methyl-1-propanesulfonic) acid; and copolymers thereof such as polypropylene glycol/polyethylene glycol copolymers.

Nonpolymeric materials useful as blood absorbent enhancing agents in the present invention are also described in detail in U.S. Pat. No. 5,998,032, which is incorporated expressly herein by reference. The nonpolymeric enhancing agents are generally organic and include at least one hydrogen bond forming functionality. Such functionality may include a functional group selected from a carboxyl, a carboxylate, a carbonyl, a sulfonic acid, a sulfonamide, a sulfonate, a phosphate, a phosphoric acid, a phosphoramide, a hydroxyl, an amide, an amine, and combinations thereof.

One particularly useful family of non-polymeric blood absorbent enhancing agent includes hydroxyacids, such as lactic acid, tartaric acid, ascorbic acid, citric acid, salicylic acid, their salts, and combinations thereof. One example of a salt of a hydroxy acid is sodium lactate. In accordance with the present invention, the blood absorbent enhancing agent is applied to the fibers to provide a total amount of the agent in the web ranging from about 1 to about 40 weight % based on the weight of the fibers within the web. As described above, the blood absorbent enhancing agent may be distributed throughout the web so as to have a non-uniform distribution. The particular design of this non-uniform distribution will depend upon the ultimate end use of the absorbent structure; however, in the context of sanitary napkins, the amount of the blood absorbent enhancing agent in one embodiment is greater near a first surface of the web and less near a second surface of the web that is spaced apart from the first surface. Alternatively, the distribution of the blood absorbent enhancing agent can be just the opposite wherein the amount is less near the first surface and greater near the second surface. By providing regions within the web that have a greater amount of the blood absorbent enhancing agent, it is possible to expose the incoming blood to a given amount of the blood enhancing agent without requiring that such amount of blood absorbent enhancing agent be present uniformly throughout the web. For example, when X represents the amount of blood absorbent enhancing agent needed to modify the blood absorbent properties of the web of cellulose fibers, such amount can be provided at a first surface, while other portions of the web can contain an amount less than X. This has several advantages including reducing the total amount of blood enhancing agent in the web which leads to reductions in manufacturing costs.

In a preferred embodiment, the amount of blood enhancing agent increases through the web in a direction away from the surface of the absorbent structure that is adjacent to the wearer's skin. In other words, the surface of the absorbent web that is adjacent to the wearer's skin includes an amount of the blood absorbent enhancing agent that is less than the amount of blood absorbent enhancing agent that is present at the opposite surface which is further removed from the wearer's skin. This particular configuration of a non-uniform distribution of the blood enhancing agent in the web promotes coagulation of the blood at a location that is further removed from the surface of the wearer's skin as compared to where coagulation would occur if the amount of the blood absorbent enhancing agent was greater near the surface of the web adjacent to the wearer's skin.

The degree of the difference between the amount of the blood enhancing agent adjacent the first surface and the amount of the blood enhancing agent adjacent the second surface will vary depending upon the particular end application. The amounts should be chosen so that coagulation of the blood occurs at a desired location within the web. For example, in instances where coagulation of the blood adjacent the surface of the wearer is desired, the amount of blood enhancing agent in the web present adjacent this surface would be higher than the amount of the blood enhancing agent present at the surface that is furthest removed from the wearer's skin.

The blood absorbent enhancing agent can be applied using sprayers, rollers, or immersion applicators that are designed to provide a non-uniform distribution of the blood absorbent enhancing agent in the web. Alternatively, the web of fibers can be formed from different lots of fibers that have been pretreated with different amounts of the blood absorbent enhancing agent.

Referring back to FIGS. 1 and 2, web 10 can include superabsorbent materials to absorb the aqueous portions of the blood. Superabsorbent materials are generally well known and come in many forms such as particles, granules, or fibers. These materials comprise polymers that swell on exposure to water and form a hydrated gel (hydrogel) by absorbing large amounts of water. Superabsorbent materials include starch graft, copolymers, crosslinked carboxymethylcellulose derivatives, and modified hydrophilic polyacrylates. Such superabsorbent materials are available from numerous commercial sources including Hoechst-Celanese of Portsmouth, Va., the Dow Chemical Company of Midland, Mich., and Stockhausen of Greensboro, N.C.

Referring to FIG. 3, web 10 can be combined with an additional web of fibers 20 as illustrated in FIG. 3. Web 20 may include superabsorbent materials, and the fibers of web 20 may or may not be treated with chemicals to modify their properties. Chemicals that may be applied to the fibers of web 20 include the blood absorbent enhancing agents described above as well as the other chemicals described above. Web 20 can be compressed to a density that is greater than the density of web 10.

When combined with web 20, web 10 serves as a layer where coagulation of blood occurs with a portion of the aqueous components of the blood passing through web 10 into web 20 where the superabsorbent material absorbs and retains the aqueous components.

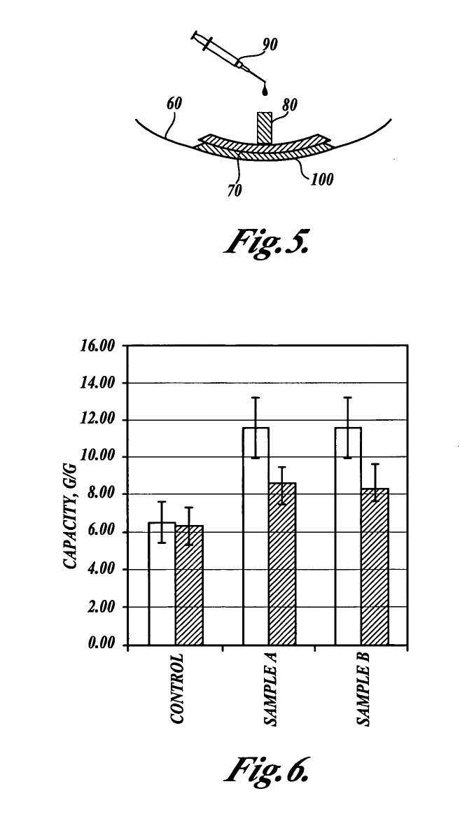

Referring to FIG. 4, in a second embodiment of the present invention, a web 30 of fibers treated with a blood absorbent enhancing agent in the same manner as described above with reference to web 10 and FIGS. 1 and 2 can be combined with an upper web 40 of fibers that serves to initially acquire the menstrual fluid. Upper web 40 includes treated or untreated cellulosic or synthetic fibers such as those described above. Upper web 40 can be formed by conventional means such as airlaying. The fibers of web 40 are bonded together as described above. Preferably, web 40 comprises crosslinked cellulose fibers that have been bonded together using wet strength resins, thermobondable fibers, or bonding mediums such as those described in U.S. Pat. No. 5,837,627, the subject matter of which is expressly incorporated herein by reference. Web 40 has a density that is lower than the underlying web 30. For example, upper web 40 has a density ranging from about 0.03 to about 0.08 g/cm³ while web 30 has a density ranging from about 0.08 to about 0.60 g/cm³.

In embodiments where web 30 does not include superabsorbent material, an additional web 50 can be provided on the side of web 30 opposite web 40. Web 50 includes fibers and superabsorbent material. Web 50 absorbs the aqueous component of the blood that passes through web 30.

Certain aspects of the present invention will now be illustrated by reference to the following examples, which are not intended to limit the scope of the present invention.

### EXAMPLE 1

This example illustrates how structures of the present invention exhibit improved absorbance under load (AUL) and capacity characteristics compared to a commercially available feminine hygiene product.

The commercially available feminine hygiene product comprises a top layer made from a synthetic material with holes punched into it. The top layer has a basis weight of 73.5 g/m² and a density of 0.12 g/cm³. The bottom storage layer has a basis weight of 152 g/m² and a density of 0.13 g/cm³. The bottom storage layer includes airlaid cellulose fluff bonded with latex. The bottom layer also includes about 20 weight % SAP based on the weight of the fibers. The overall basis weight is 225 g/m².

As an example of the present invention, a two-layer structure having a total basis weight of 200 g/m² is formed from an upper web and a lower web. The upper web has a basis weight of 100 g/m² and the lower web has a basis weight of 100 g/m². Table 1 sets forth the composition of the respective fibers used in the respective layers.

TABLE 1
Fiber Compositions

| | |
|---|---|
| A | Standard cellulose fluff pulp available from Weyerhaeuser |
| | Company, Federal Way, Washington. |
| B | Fiber A with 2.5 wt % add on of lactic acid. |
| C | Fiber A treated with a solution of 70% sorbitol, 30% propylene |
| | glycol, and H₂O @ 76% solids. |

The lactic acid on Fiber B is provided by applying an aqueous solution of 28 wt % of an 88% solids solution of lactic acid, 27 wt % of a 60% solids solution of sodium lactate and 45 wt % of a 99% solids solution of propylene glycol onto Fiber A at a loading of .9 wt. % based on the weight of the fiber

Fiber C is provided by applying an aqueous solution having a solids content of 76% that includes 70 wt % of a 70% solids solution of orbital, 30 wt % of a 99% solids solution of propylene glycol and water onto Fiber A at a loading of 9 wt % based on the weight of the fibers.

The densities of the upper web and lower web fall within the ranges set forth below in Table 2. The fibers used to form the upper web and the lower web are also set forth below in Table 2 as well. As indicated in Table 1, in certain samples, superabsorbent material is present in the second web.

TABLE 2

| | | SAMPLE A | SAMPLE B |
|---|---|---|---|
| Upper Web 1 | Fiber | Fiber B | Fiber B |
| | Basis Weight (g/m²) | 100 | 100 |
| | Density (g/cm³) | 0.03-0.1 | 0.03-0.1 |
| Lower Layer 2 | Fiber | Fiber A | Fiber C |
| | Basis Weight (g/m²) | 100 | 100 |
| | Density(g/cm³) | 0.25 | 0.25 |
| | SAP | 20 wt % | 20 wt % |

Each web was formed as an airlaid pad on a 35.5 cm by 35.5 cm pad former. Sufficient fiber was used to obtain the appropriate basis weight and was separated into four piles. After one pile of fiber was added to the pad former, the sheet was rotated 90 degrees. Then, the next pile of fibers was added. This was continued until all the piles were added to the former. Samples measuring 6 cm by 20 cm were cut from the airlaid pads and matched with other cut samples based on the designs of Table 1. Densification was achieved by compressing the sample to a maximum of 7,500 psi on a laboratory Carver press. The final samples were prepared by matching the top and bottom layers in accordance with Table 1 and adding a nonwoven top sheet. The top sheet used was cut from a Procter & Gamble commercial sanitary napkin product.

Absorbency under load (AUL) and retention were evaluated using an apparatus that simulates a product used in a standing position. The apparatus is illustrated in FIG. 5. The apparatus includes an arcuate saddle frame 60 used to hold a sample and an 850 g dosing weight 70. The dosing weight includes a curvature that is congruent with the curvature of the saddle frame 60. The dosing weight applies a load of 0.09 psi to a sample that is placed on the saddle frame beneath the dosing weight. Dosing weight 70 includes a dosing tube 80 extending perpendicularly from the surface of the dosing weight opposite the surface, which contacts the sample. The dosing tube 80 allows blood to be added to the underlying sample.

Blood is added to the dosing tube 80 via a syringe 90 at a rate of 1 ml every 10 seconds. The blood is added at this rate until sample 100 is obviously leaking from the sides. At the point of leakage, dosing is stopped and the sample is allowed to stand for 30 minutes. After the waiting period, dosing of blood resumes until leakage again occurs. The sample is allowed to stand for an additional 5 minutes prior to weighing the sample. The absorbency under load in grams per gram is obtained by taking this weight, subtracting the dry weight of the sample, and then dividing by the dry weight of the sample.

Retention is obtained by taking the absorbency under load wetted sample and squeezing out the free liquid under pressure of 1 psi. This is done in a Büchner funnel with a controlled vacuum delivering the desired load. The sample is then weighed after the free liquid is removed. Retentions in grams per gram is calculated by taking this weight, subtracting the dry weight of the sample, and dividing by the dry weight of the sample. Each sample was tested in triplicate for both absorbency under load and retention. The results are graphically illustrated in FIG. 6.

Absorbent structures comprising a web of wood pulp fibers treated with lactic acid combined with an undensified web or a densified web of wood pulp fibers containing a superabsorbent material exhibit superior absorbency under load and retention properties compared to the control.

While preferred embodiment of the invention has been illustrated and described, it will be appreciated that various changes can be made therein without departing from the spirit and scope of the invention.

1. An absorbent structure for use in an article for absorbing blood, the absorbent structure comprising:
fibers formed into a first web, the first web having a first surface and a second surface spaced from the first surface;
blood absorbent enhancing agent within the web, the blood absorbent enhancing agent present in a first amount adjacent the first surface and a second amount adjacent the second surface, the first amount being unequal to the second amount.

2. The absorbent structure of claim 1, wherein the total amount of the blood absorbent enhancing agent within the web ranges from about 1% to about 40% based on the weight of the fibers.

3. The absorbent structure of claim 1, wherein the blood absorbent enhancing agent is lactic acid.

4. The absorbent structure of claim 1, wherein the blood absorbent enhancing agent comprises a mixture of lactic acid and sodium lactate.

5. The absorbent structure of claim 1, wherein the blood absorbent enhancing agent is sodium lactate.

6. The absorbent structure of claim 3, wherein the total amount of lactic acid in the web ranges from about 1% to about 40% based on the weight of the fibers.

7. The absorbent structure of claim 1 further comprising a superabsorbent material.

8. The absorbent structure of claim 1, wherein the fibers are cellulose fibers.

9. The absorbent structure of claim 1 further comprising a second web of fibers.

10. The absorbent structure of claim 9, wherein the fibers of the second web comprise cellulose fibers.

11. The absorbent structure of claim 10, wherein the second web is compressed to increase its density.

12. The absorbent structure of claim 11, wherein the first web is less dense than the second web.

13. The absorbent structure of claim 10, wherein the second web includes superabsorbent material.

14. An absorbent structure for use in an article for absorbing blood, the absorbent structure comprising:
a first web comprising fibers that are bonded together, the first web having a first density; and
a second web comprising fibers and a blood absorbent enhancing agent.

15. The absorbent structure of claim 14, wherein the blood absorbent enhancing agent is lactic acid.

16. The absorbent structure of claim 14, wherein the blood absorbent enhancing agent comprises a mixture of lactic acid and sodium lactate.

17. The absorbent structure of claim 14, wherein the blood absorbent enhancing agent is sodium lactate.

18. The absorbent structure of claim 14, wherein the first web includes wet strength resins.

19. The absorbent structure of claim 14, wherein the first web includes thermobondable fibers.

20. The absorbent structure of claim 14, wherein the first web has a density ranging from about 0.03 to about 0.2 g/cm³.

21. The absorbent structure of claim 20, wherein the first web has a density ranging from about 0.03 to about 0.08 g/cm³.

22. The absorbent structure of claim 14, wherein the second web has been compressed to increase its density to a second density greater than the first density.

23. The absorbent structure of claim 14, wherein the first web comprises cellulose fibers.

24. The absorbent structure of claim 14, wherein the second web comprises cellulose fibers.

25. The absorbent structure of claim 14, wherein the second web includes a superabsorbent material.

26. The absorbent structure of claim 14, wherein the second web has a first surface and a second surface spaced from the first surface, the blood absorbent enhancing agent present in a first amount adjacent the first surface and a second amount adjacent the second surface, the first amount being unequal to the second amount.

27. The absorbent structure of claim 21, wherein the second web has a density ranging from about 0.08 to about 0.6 g/cm³.
